(19) United States
(12) Patent Application Publication
(10) Pub. No.: US 2025/0014714 A1
(43) Pub. Date: Jan. 9, 2025

(54) DIETARY SUPPLEMENT COMPOUNING ASSISTANCE SYSTEM AND DIETARY SUPPLEMENT COMPOUNDING ASSISTANCE METHOD

(71) Applicant: dricos, Inc., Tokyo (JP)

(72) Inventor: Yasuhiro Take, Tokyo (JP)

(21) Appl. No.: 18/755,413

(22) Filed: Jun. 26, 2024

(30) Foreign Application Priority Data
Jun. 26, 2023 (JP) 2023-104111

(51) Int. Cl. G16H 20/60; G16H 10/60; G16H 20/30

(52) U.S. Cl. CPC G16H 20/60; G16H 10/60; G16H 20/30

(57) ABSTRACT

A dietary supplement compounding assistance system 1 includes: a first acquisition unit 271 which acquires information relating to exercise to be performed by a user; a second acquisition unit 272 which acquires an objective relating to health of the user; a nutrient information memory 12I which stores nutrient information having a correlation with the information relating to exercise and a correlation with the objective relating to health; a search unit 281 which searches the nutrient information memory 12I for nutrient information having a correlation with the information relating to exercise and a correlation with the objective relating to health; a dietary supplement determination unit 282 which determines types of nutrients and compounding amounts of the nutrients based on the searched-for and obtained nutrient information; and a display 252 which presents the searched-for and obtained nutrient information.

### TECHNICAL FIELD

The present disclosure relates to a dietary supplement compounding assistance system and a dietary supplement compounding assistance method.

### BACKGROUND ART

In recent years, as health consciousness rises, the supplement market is expanding as a means for maintaining health. The use of fitness centers, including sports gyms, is also expanding as a way to maintain health. By addressing health from both nutritional and exercise perspectives, one can more effectively maintain and enhance overall well-being.

Nutrition is closely relating to bodily functions, and a correlation can be observed between the optimal composition of supplement ingredients and the type of exercise the user engages in. Especially when the user has set specific goals through exercise, such as dieting or increasing muscle strength, or when there are ideal targets for metrics like body fat percentage or muscle mass, the likelihood of achieving the user's health-related goals or ideal state is significantly enhanced when both exercise and nutrition are optimized together. However, providing the user with the optimal supplements while considering the correlation between nutrition and exercise requires specialized knowledge.

For example, PTL 1 discloses a dietary supplement compounding assistance system that determines a composition of dietary supplements by comparing information such as information relating to a user and biological information and external environment information acquired by sensors and devices with the user's objective of taking dietary supplement, comparing with an ideal dietary supplement intake, and/or comparing with an ideal state such as the user's goal.

The dietary supplement compounding assistance system disclosed in PTL 1 can provide assistance in compounding the optimal supplements tailored to various health-related objectives and goals of the user, such as nutrient supplementation and enhancement, disease prevention, fatigue recovery, beauty, and dieting. However, the technology disclosed in PTL 1 cannot assist the user in achieving health-related purposes and goals by promoting the intake of more effective supplements based on the correlation between exercise and nutrition.

### CITATION LIST

### Patent Literature

PTL 1: Japanese Patent Laid-Open No. 2017-207819

### SUMMARY OF INVENTION

### Technical Problem

According to conventional technologies, it has been challenging to provide more effective assistance for compounding dietary supplements based on the correlation between exercise and nutrition in order to achieve and enhance health.

The present disclosure has been made to solve the problem described above and an object thereof is to provide more effective assistance for compounding dietary supplements based on the correlation between exercise and nutrition in order to achieve and enhance health.

### Solution to Problem

In order to solve the problem described above, a dietary supplement compounding assistance system according to the present disclosure includes: a first acquisition unit which acquires information relating to exercise to be performed by a user; a second acquisition unit which acquires an objective relating to health of the user; a first memory which stores nutrient information having a correlation with the information relating to exercise and having a correlation with the objective relating to health; a search unit which searches the first memory for nutrient information having a correlation with the information relating to exercise and having a correlation with the objective relating to health; a calculator which determines a type of nutrient and a compounding amount of the nutrient based on the searched-for and obtained nutrient information; and a presenter which presents the searched-for and obtained nutrient information.

Further, the dietary supplement compounding assistance system according to the present disclosure may further include a supplier which supplies a dietary supplement containing the compounding amount of the nutrient determined by the calculator, wherein the presenter may present information relating to a supply of the dietary supplement.

Yet further, the dietary supplement compounding assistance system according to the present disclosure may further include a physical change calculator which calculates, based on physical information of the user, lifestyle habit information, and biological information and external environment information acquired by a sensor, an amount of change in the physical information of the user before and after the user engages in the exercise and takes in the dietary supplement, wherein the presenter may present the amount of change in the physical information of the user.

Yet further, the dietary supplement compounding assistance system according to the present disclosure may further include: a second memory which stores the information relating to the exercise of the user, the objective relating to health, information relating to a supply of the dietary supplement, the amount of change in the physical information, and physical characteristics of the user in association with identification information of the user; a first comparator which compares the information relating to exercise and the amount of change in the physical information among a plurality of users whose physical characteristics have a value within a certain range from a value of the physical characteristics of the user, and who have received a supply of the dietary supplement; and a generator which generates exercise assistance information for the exercise to be performed by the user in accordance with the objective relating to health of the user based on a result of the comparison by the first comparator, wherein the presenter may present the exercise assistance information to the user.

Yet further, the dietary supplement compounding assistance system according to the present disclosure may further include: a second comparator which compares an amount of change in the physical information of the user having received a supply of the dietary supplement by the supplier and an amount of change in the physical information of a user not having received a supply of the dietary supplement; and an assessment unit which calculates, based on a result of the comparison by the second comparator, a degree of contribution of a supply of the dietary supplement relative to the amount of change in the physical information of the user having received the supply of the dietary supplement and which assesses the degree of contribution, wherein the presenter may present a result of the assessment by the assessment unit.

Yet further, the dietary supplement compounding assistance system according to the present disclosure may further include an inference unit which infers time during which the user had engaged in the exercise based on information on a time point at which the first acquisition unit had acquired the information relating to exercise, wherein the presenter may present the inferred time during which the user had engaged in the exercise.

Yet further, the dietary supplement compounding assistance system according to the present disclosure may further include a reception which receives a review relating to a supply of the dietary supplement from the user having received the supply of the dietary supplement.

Yet further, in the dietary supplement compounding assistance system according to the present disclosure, the search unit may search the first memory for the nutrient information in consideration of the review received by the reception.

In order to solve the problem described above, a dietary supplement compounding assistance method according to the present disclosure includes: a first step of acquiring information relating to exercise to be performed by a user; a second step of acquiring an objective relating to health of the user; a third step of storing nutrient information having a correlation with the information relating to exercise, and having a correlation with the objective relating to health in a first memory; a fourth step of searching the first memory for nutrient information having a correlation with the information relating to exercise, and having a correlation with the objective relating to health; a fifth step of determining a type of nutrient and a compounding amount of the nutrient based on the searched-for and obtained nutrient information; and a sixth step of presenting the searched-for and obtained nutrient information.

Further, the dietary supplement compounding assistance method according to the present disclosure may further include a seventh step of supplying a dietary supplement containing the compounding amount of the nutrient determined in the fifth step, wherein in the sixth step, information relating to a supply of the dietary supplement may be presented.

Yet further, the dietary supplement compounding assistance method according to the present disclosure may further include an eighth step of calculating, based on physical information of the user, lifestyle habit information, and biological information and external environment information acquired by sensors, an amount of change in the physical information of the user before and after the user engages in the exercise and takes in the dietary supplement, wherein in the sixth step, the amount of change in the physical information of the user may be presented.

Yet further, the dietary supplement compounding assistance method according to the present disclosure may further include: a ninth step of storing, in a second memory, the information relating to the exercise of the user, the objective relating to health, information relating to a supply of the dietary supplement, the amount of change in the physical information, and physical characteristics of the user in association with identification information of the user; a tenth step of comparing the information relating to exercise and the amount of change in the physical information among a plurality of users whose physical characteristics have a value within a certain range from a value of the physical characteristics of the user, and who have received a supply of the dietary supplement; and an eleventh step of generating exercise assistance information for the exercise to be performed by the user in accordance with the objective relating to health of the user based on a result of the comparison in the tenth step, wherein in the sixth step, the exercise assistance information may be presented to the user.

Yet further, the dietary supplement compounding assistance method according to the present disclosure may further include: a twelfth step of comparing an amount of change in the physical information of the user having received a supply of the dietary supplement in the seventh step and an amount of change in the physical information of a user not having received a supply of the dietary supplement; and a thirteenth step of calculating a degree of contribution of a supply of the dietary supplement relative to the amount of change in the physical information of the user having received the supply of the dietary supplement based on a result of the comparison in the twelfth step and assessing the degree of contribution, wherein in the sixth step, a result of the assessment in the thirteenth step may be presented.

Yet further, the dietary supplement compounding assistance method according to the present disclosure may further include a fourteenth step of inferring time during which the user had engaged in the exercise based on information on a time point at which the information relating to exercise had been acquired in the first step, wherein in the sixth step, the inferred time during which the user had engaged in the exercise may be presented.

Yet further, the dietary supplement compounding assistance method according to the present disclosure may further include a fifteenth step of receiving a review relating to a supply of the dietary supplement from the user having received the supply of the dietary supplement.

Yet further, in the dietary supplement compounding assistance method according to the present disclosure, in the fourth step, the nutrient information may be searched for from the first memory in consideration of the review received in the fifteenth step.

### Advantageous Effects of Invention

According to the present disclosure, nutrient information having a correlation with information relating to exercise, and having a correlation with an objective relating to health is searched for from a first memory, and a type of nutrient and a compounding amount of the nutrient are determined based on the searched-for and obtained nutrient information. As a result, more effective assistance can be achieved for compounding dietary supplements based on the correlation between exercise and nutrition in order to achieve and enhance health.

### BRIEF DESCRIPTION OF DRAWINGS

FIG. 1 is a block diagram showing a configuration of a dietary supplement compounding assistance system according to an embodiment of the present disclosure;

FIG. 2 is a block diagram showing a configuration of a storage of a management server included in the dietary supplement compounding assistance system according to the present embodiment;

FIG. 3 is a block diagram showing a configuration of the management server included in the dietary supplement compounding assistance system according to the present embodiment;

FIG. 4 is a block diagram showing a configuration of a user device included in the dietary supplement compounding assistance system according to the present embodiment;

FIG. 5 is a block diagram showing a configuration of a dietary supplement supply device included in the dietary supplement compounding assistance system according to the present embodiment;

FIG. 6 is an external perspective view showing a configuration of the dietary supplement supply device included in the dietary supplement compounding assistance system according to the present embodiment;

FIG. 7 is a front view of the dietary supplement supply device included in the dietary supplement compounding assistance system according to the present embodiment;

FIG. 8 is a diagram showing an example of a configuration of a user information table included in the dietary supplement compounding assistance system according to the present embodiment;

FIG. 9 is a diagram showing an example of a configuration of a user history table included in the dietary supplement compounding assistance system according to the present embodiment;

FIG. 10 is a diagram showing an example of a configuration of a dispense history table included in the dietary supplement compounding assistance system according to the present embodiment;

FIG. 11 is a diagram showing an example of a configuration of a dietary supplement supply device management table included in the dietary supplement compounding assistance system according to the present embodiment;

FIG. 12 is a diagram showing an example of a configuration of a cartridge management table included in the dietary supplement compounding assistance system according to the present embodiment;

FIG. 13 is a diagram showing an example of a dietary supplement management table included in the dietary supplement compounding assistance system according to the present embodiment;

FIG. 14 is a diagram showing an example of a configuration of an exercise information table included in the dietary supplement compounding assistance system according to the present embodiment;

FIG. 15 is a diagram showing an example of a configuration of an objective information table included in the dietary supplement compounding assistance system according to the present embodiment;

FIG. 16 is a diagram showing an example of a nutrient information table included in the dietary supplement compounding assistance system according to the present embodiment;

FIG. 17 is a diagram showing an example of a physical information table included in the dietary supplement compounding assistance system according to the present embodiment;

FIG. 18 is a diagram showing an example of a configuration of an exercise history table included in the dietary supplement compounding assistance system according to the present embodiment; and

FIG. 19 is a sequence diagram for explaining operations of the dietary supplement compounding assistance system according to the present embodiment.

### DESCRIPTION OF EMBODIMENT

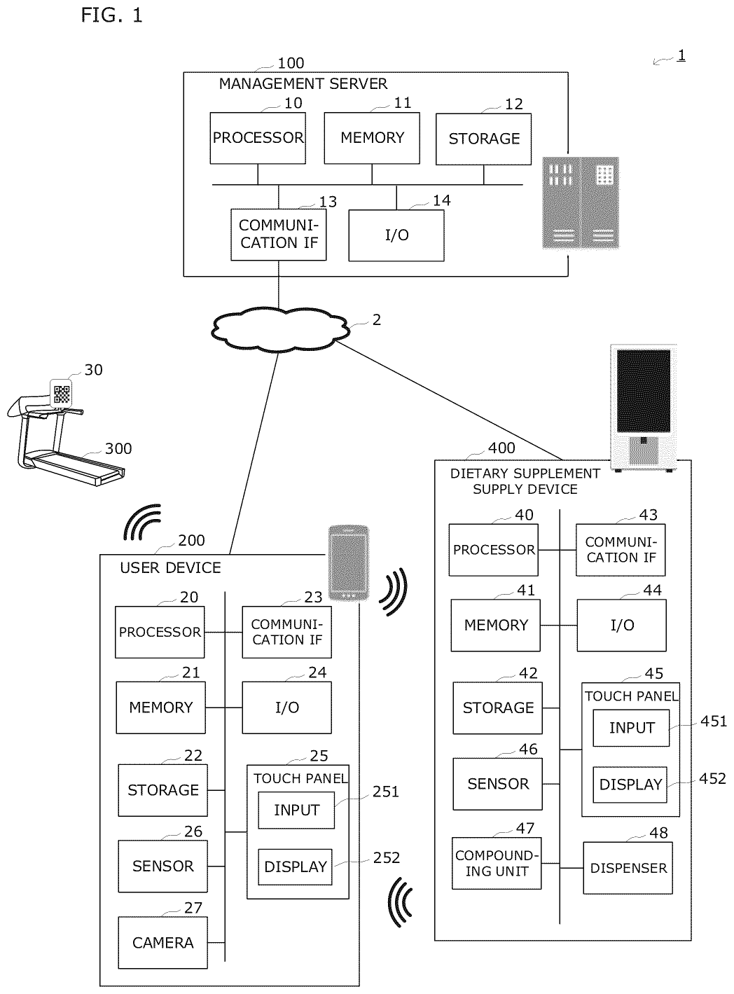

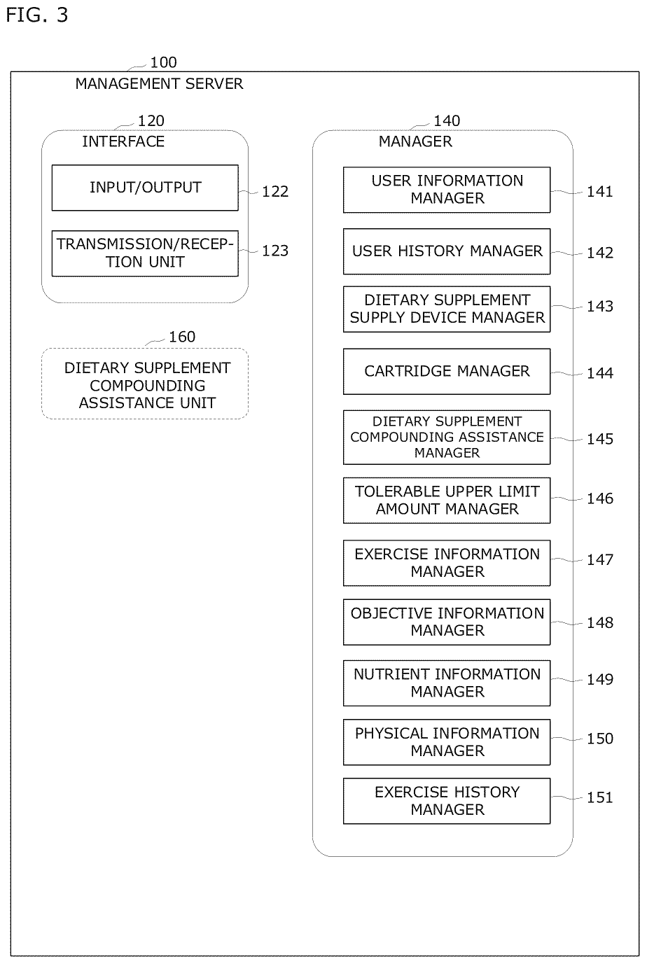

Hereinafter, a preferred embodiment of the present disclosure will be described in detail with reference to FIGS. 1 to 19.

### [Configuration of Dietary Supplement Compounding Assistance System]

First, an overview of a dietary supplement compounding assistance system 1 according to an embodiment of the present disclosure will be described. The dietary supplement compounding assistance system 1 acquires information relating to exercise to be performed by a user and an objective relating to health of the user, searches a nutrient information memory 12I (the first memory) for nutrient information that has a correlation with the acquired information relating to exercise and also has a correlation with the acquired objective relating to health, determines types and compounding amounts of nutrients based on the obtained nutrient information, and causes a user device 200 to display the nutrient information. In addition, the dietary supplement compounding assistance system 1 supplies a dietary supplement from a dietary supplement supply device 400 based on the types and the compounding amounts of nutrients.

As shown in FIG. 1, for example, the dietary supplement compounding assistance system 1 includes a management server 100, the user device 200, a fitness device 300, and the dietary supplement supply device 400. For example, the management server 100, the user device 200, and the dietary supplement supply device 400 are connected to each other via a network 2 such as the Internet. The network 2 may include a wireless network constructed by wireless base stations (not illustrated).

While the user device 200 and the dietary supplement supply device 400 are connected to each other via the network 2, the user device 200 and the dietary supplement supply device 400 are also connected to each other via a near field communication network such as Bluetooth Low Energy (BLE, registered trademark; the same applies hereafter), RFID, or NFC. In addition, the user device 200 and the fitness device 300 may also be connected to each other via a near field communication network such as BLE, RFID, or NFC.

Alternatively, a configuration may be adopted in which the management server 100 and the dietary supplement supply device 400 are not connected to each other via the network 2 but the management server 100 communicates with the dietary supplement supply device 400 by near field communication via the user device 200.

In addition, a configuration may be adopted in which the dietary supplement supply device 400 is operated as an offline stand-alone terminal.

### [Configuration of Management Server]

The management server 100 includes a processor 10, a memory 11, a storage 12, a communication interface 13, and an input/output interface 14 which are electrically connected to each other via a bus.

The management server 100 is an information processing apparatus that is used when a system administrator or the like maintains and manages a dietary supplement compounding assistance service. For example, a general-purpose computer such as a work station or a personal computer can be used. The management server 100 maintains basic information of service, master information, and management information relating to the dietary supplement compounding assistance service.

The processor 10 is, for example, a CPU (Central Processing Unit) and realizes necessary processing by executing a program or the like stored in the storage 12 and deployed on the memory 11.

The memory 11 is, for example, a volatile storage device such as a DRAM (Dynamic Random Access Memory).

The storage 12 is, for example, a non-volatile storage device such as an HDD (Hard Disc Drive). The storage 12 may store an authentication program of a user, a dietary supplement compounding assistance program, or the like and may be provided with areas corresponding to memories 12A to 12K to be described later, and the memories 12A to 12K may be constructed by a database such as a group of tables 504 to 564 to be described later.

The communication interface 13 is an interface circuit for providing a network connection between the management server 100 and various external electronic equipment.

The input/output (I/O) 14 is an information input device such as a mouse or a keyboard or a liquid crystal display.

### [Configuration of Storage]

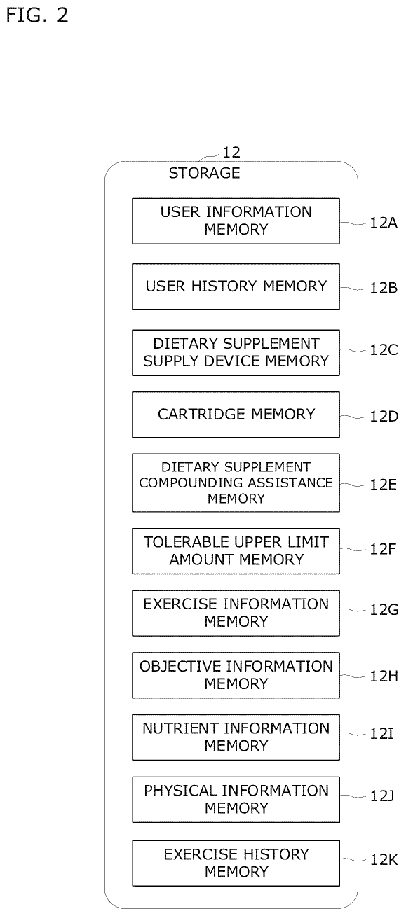

A configuration of the storage 12 included in the management server 100 will now be described with reference to FIG. 2. For example, the storage 12 includes a user information memory 12A, a user history memory 12B, a dietary supplement supply device memory 12C, a cartridge memory 12D, a dietary supplement compounding assistance memory 12E, a tolerable upper limit amount memory 12F, an exercise information memory 12G, an objective information memory 12H, the nutrient information memory 12I, a physical information memory 12J, and an exercise history memory 12K. A second memory includes the exercise information memory 12G, the objective information memory 12H, the user history memory 12B, and the physical information memory 12J.

The user information memory 12A stores basic information of a user such as user identification information.

The user history memory 12B stores usage history of dietary supplements by the user.

The dietary supplement supply device memory 12C stores an installation location, a status, and the like of the dietary supplement supply device 400.

The cartridge memory 12D stores serial numbers and dietary supplement information of cartridges.

The dietary supplement compounding assistance memory 12E stores various pieces of information necessary for implementing dietary supplement compounding assistance.

The tolerable upper limit amount memory 12F stores a tolerable upper limit amount of a dietary supplement.

The exercise information memory 12G stores information relating to exercise to be performed by the user. For example, the exercise information memory 12G can store contents of exercise associated with a type of the fitness device 300.

The objective information memory 12H stores an objective relating to health of the user. While details will be provided later, the objective relating to health of the user is an objective regarding the maintenance of health, improvement of physical strength, or lifestyle that can be optionally set by the user and examples include “dieting”, “building muscle strength”, and “improving endurance”.

The nutrient information memory 12I stores nutrient information that has a correlation with information relating to exercise to be performed by the user, and with an objective relating to health. The nutrient information memory 12I stores information in which details of each exercise and an objective relating to health of the user are associated with nutrients based on correlations calculated in advance or, more specifically, a correlation between a content of each exercise and nutrients and a correlation between the objective relating to health of the user and nutrients. The correlation between exercise and nutrients, as well as the correlation between the user's health objectives and nutrients, are calculated in advance by, for example, multivariate analysis such as principal component analysis or by learning characteristics among variables using a machine learning algorithm.

The physical information memory 12J stores physical information, physical characteristics, lifestyle habit information, and the like for each user. Physical information includes weight, body-fat percentage, and muscle mass of the user which are elements that may change as the user engages in exercise or takes in nutrition. In addition, physical characteristics of the user include elements in addition to physical information which generally remain unchanged regardless of exercise and intake of nutrition. Lifestyle habit information includes an amount of sleep by the user, a frequency and an amount of drinking, whether or not the user smokes, indices relating to stress, information relating to eating habits, a presence or absence of illnesses, and information relating to exercise habits. The exercise history memory 12K stores a history of exercises performed by the user.

### [Configuration of User Device]

Returning to FIG. 1, the user device 200 includes a processor 20, a memory 21, a storage 22, a communication interface 23, an input/output (I/O) 24, a touch panel 25, a sensor 26, and a camera 27.

The user device 200 acquires information relating to exercise to be performed by the user and an objective relating to health of the user. In addition, the user device 200 can acquire nutrient information having been searched for and obtained from the nutrient information memory 12I of the management server 100 via the network 2 and display the nutrient information on a display 252. As the user device 200, for example, a mobile device that can be carried around by the user such as a smartphone, a feature phone, a PDA, and a tablet computer can be used.

The processor 20, the memory 21, the storage 22, and the I/O 24 can be configured in a similar manner to their counterparts included in the management server 100 described above.

The communication interface 23 is an interface circuit for providing a network connection between the user device 200 and various external electronic equipment. A configuration may be adopted in which, in addition to providing connections to the management server 100 and to the dietary supplement supply device 400 via the network 2, the communication interface 23 provides connections to the fitness device 300 and the dietary supplement supply device 400 by near field communication.

The touch panel 25 is an electronic component which combines an input 251 including a touch pad with the display 252 including a liquid crystal display. The input 251 has a function of detecting information on a position on a screen at which a physical contact operation such as a touch operation by the user has been input and outputting a signal externally. The touch panel 25 may be any system including a capacitance system and a resistive system.

The sensor 26 includes, as built-in sensors, an acceleration sensor, a vibration sensor, a gyroscope sensor, a geomagnetic sensor, a proximity sensor, an illuminance sensor, a pressure sensor, the GPS, a thermometer, and a hygrometer.

The camera 27 can convert an optical signal into an image signal and generate a moving image or a still image. The camera 27 captures a still image of a two-dimensional code 30 attached to the fitness device 300, performs decoding of the captured two-dimensional code 30 with the processor 20, and detects information such as a type of the fitness device 300.

### [Configuration of Fitness Device 300]

The fitness device 300 is a device installed in a fitness facility such as a sports gym, a rehabilitation facility, or the like and examples thereof include wide varieties of training machines such as walking machines/treadmills, fitness bikes, steppers, abdominal machines, and chest press machines, instruments such as dumbbells and balance balls, and rehabilitation devices such as walkers.

A two-dimensional code 30, encoding information such as the type of fitness device 300, is attached to the fitness device 300. In addition to a configuration in which the two-dimensional code 30 is attached to the fitness device 300, the two-dimensional code 30 may also be provided at an entrance to the fitness facility where the fitness device 300 is installed or the like. The two-dimensional code 30 attached to the fitness device 300 is scanned when the user carrying the user device 200 uses the fitness device 300.

### [Configuration of Dietary Supplement Supply Device]

The dietary supplement supply device 400 includes a processor 40, a memory 41, a storage 42, a communication interface 43, an input/output (I/O) 44, a touch panel 45, a sensor 46, a compounding unit 47, and a dispenser 48.

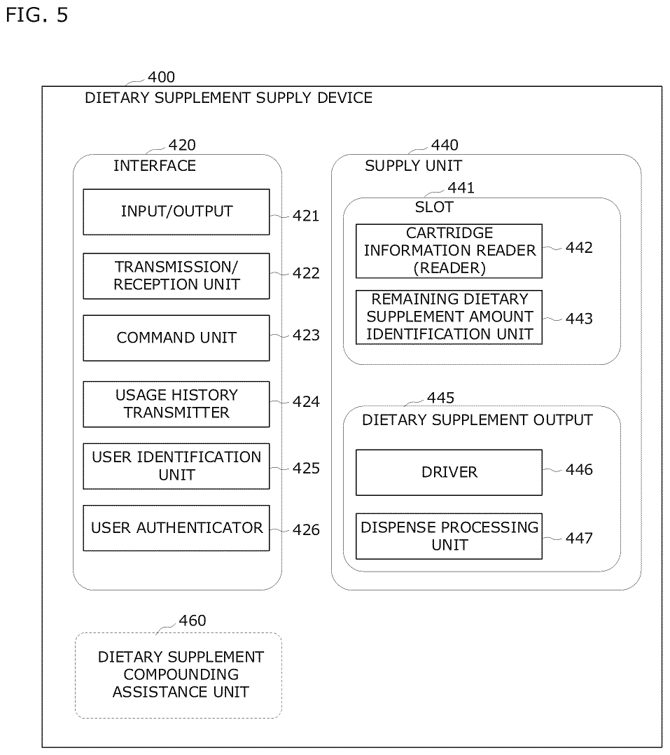

The dietary supplement supply device 400 is an apparatus which receives a dietary supplement supply instruction from the user device 200, and compounds a dietary supplement based on an instructed type and an instructed amount of the dietary supplement, and supplies the dietary supplement to the user by dispensing the dietary supplement. The dietary supplement supply device 400 has an exterior design, including an enclosure such as that shown in FIG. 5 (to be described later).

The processor 40, the memory 41, the storage 42, the communication interface 43, the I/O 44, and the touch panel 45 can be configured in a similar manner to their counterparts included in the user device 200 or the management server 100.

The sensor 46 can include a biological sensor such as an ECG sensor or a myoelectric sensor, a meteorological sensor, and the like.

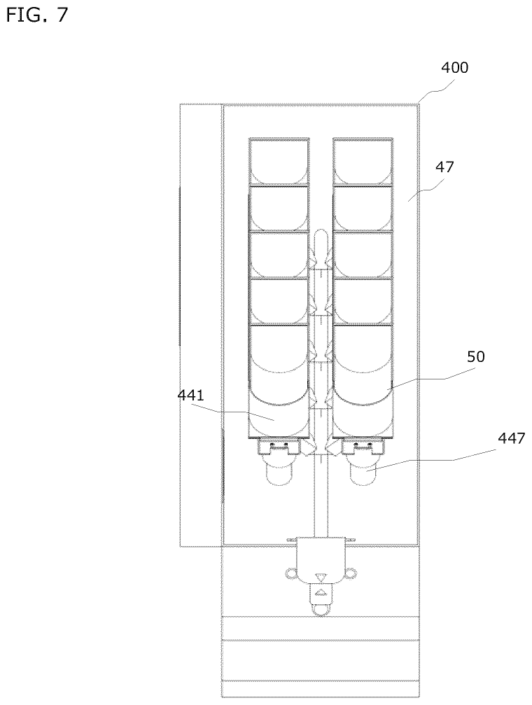

The compounding unit 47 includes a driver that provides drive in accordance with the dietary supplement supply instruction. The driver drives to rotate the mounted cartridges such as those shown in FIG. 7 so that instructed amounts of dietary supplements stored in the cartridges are extracted, and the extracted dietary supplements are compounded.

The dispenser 48 is configured to dispense and output the compounded dietary supplement externally.

### [Functional Blocks of Management Server]

FIG. 2 is a functional block diagram of the management server 100 with the hardware configuration described above. The management server 100 includes an interface 120, a manager 140, and a dietary supplement compounding assistance unit 160.

The interface 120 includes an input/output 122 and a transmission/reception unit 123.

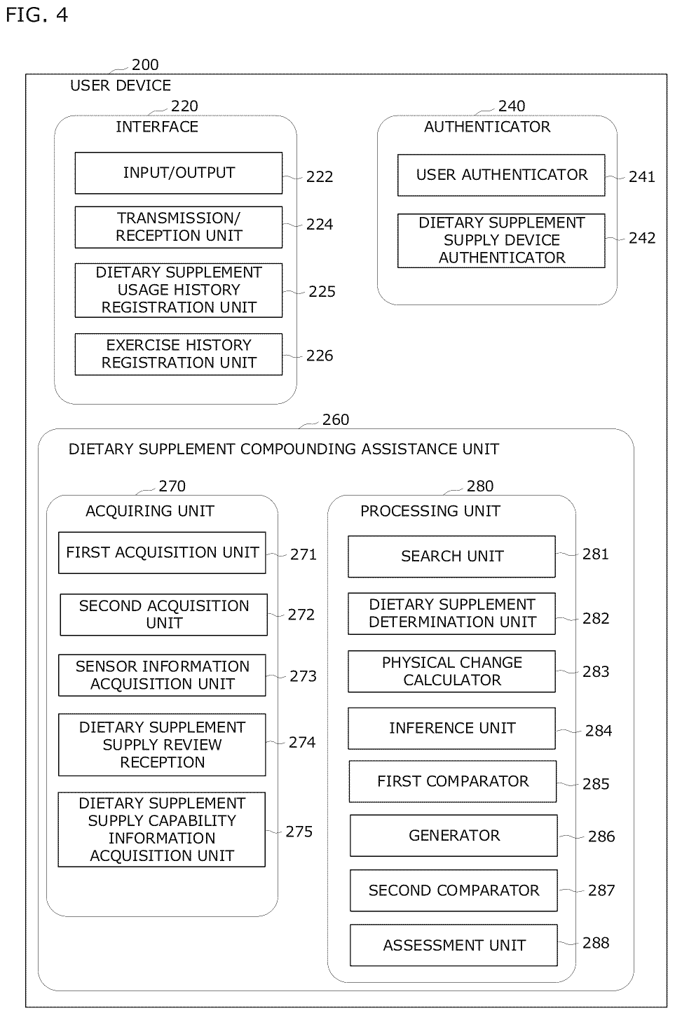

The manager 140 includes: a user information manager 141 which manages basic information of the user; a user history manager 142 which manages a usage history of the user; a dietary supplement supply device manager 143 which manages an installation location, a status, and the like of the dietary supplement supply device 400; a cartridge manager 144 which manages serial numbers and dietary supplement information of cartridges; a dietary supplement compounding assistance manager 145 which manages various pieces of information necessary for implementing dietary supplement compounding assistance; a tolerable upper limit amount manager 146 which manages a tolerable upper limit amount of dietary supplements; an exercise information manager 147 which manages information relating to exercise to be performed by the user; an objective information manager 148 which manages an objective relating to health of the user; a nutrient information manager 149 which manages nutrient information that has a correlation with the information relating to exercise to be performed by the user and has a correlation with the objective relating to health; a physical information manager 150 which manages physical information of the user; and an exercise history manager 151 which manages an exercise history of the user. The manager 140 manages basic information necessary for processing in each manager. The dietary supplement compounding assistance unit 160 (dashed line part) will be described later with reference to FIG. 4.

### [Functional Blocks of User Device]

FIG. 4 is a functional block diagram of the user device 200 with the hardware configuration described above. The user device 200 includes an interface 220, an authenticator 240, and a dietary supplement compounding assistance unit 260.

In addition to an input/output 222 and a transmission/reception unit 224, the interface 220 includes a dietary supplement usage history registration unit 225 for registering the usage history of dietary supplements by the user and the exercise history registration unit 226 for registering the history of exercise by the user at the fitness facility.

The authenticator 240 includes a user authenticator 241 which performs login authentication based on a user ID/password with the management server 100 and a dietary supplement supply device authenticator 242 which performs pairing by near field communication such as BLE with the dietary supplement supply device 400. It is to be understood that authentication systems adopted by each of the authenticators 241 and 242 are not limited to the above and any authentication system may be utilized.

The dietary supplement compounding assistance unit 260 includes an acquisition unit 270 which acquires various kinds of data from internally and externally of the device through the interface 220 and a processing unit 280 which refers to various stored tables and performs information processing.

The acquisition unit 270 includes: a first acquisition unit 271 which acquires information relating to exercise to be performed by the user; a second acquisition unit 272 which acquires an objective relating to health of the user; a sensor information acquisition unit 273 which acquires biological information and external environment information from sensors; a dietary supplement supply review reception 274 (a reception) which accepts, after a supply of a dietary supplement, a review relating to the supply of dietary supplement from the user; and a dietary supplement supply capability information acquisition unit 275 which receives dietary supplement supply capability information with respect to each cartridge included in the dietary supplement supply device 400 from the dietary supplement supply device 400.

The information relating to exercise to be performed by the user is information indicating a type of exercise to be performed by the user using the fitness device 300. For example, information relating to exercise includes the type of the fitness device 300 detected by decoding the two-dimensional code 30 attached to the fitness device 300 such as the “running” exercise performed by the user using a “treadmill”. Alternatively, a configuration may be adopted in which information relating to the “running” exercise performed using the fitness device 300 is encoded in the two-dimensional code 30 in advance and the type of the exercise is directly read from the two-dimensional code 30.

The first acquisition unit 271 can acquire from the exercise information memory 12G (an exercise information table 560 to be described later) included in the storage 12, as information relating to exercise, the type of exercise “running” associated with “treadmill” from the information read from the two-dimensional code 30.

The objective relating to health of the user that is acquired by the second acquisition unit 272 is an objective relating to the maintenance or promotion of health or lifestyle which the user desires to improve or enhance through various actions such as engaging in exercise, taking in nutrients in the form of meals or dietary supplements, and managing rest and stress. The objective relating to health of the user includes long-term goals that are often achieved by establishing habits such as regular exercise, which lead to gradual change in the user's body over time.

In the present embodiment, information relating to exercise to be performed by the user which is acquired by the first acquisition unit 271 indicates exercise performed by the user for achieving the objective relating to health. A specific example would be a case where the user desires to achieve the objective relating to health such as “dieting”, “improving endurance”, or “building muscle strength” through “running”.

For example, the second acquisition unit 272 can acquire the objective relating to health of the user by receiving, with the input 251, an input operation by the user on the touch panel 25.

The processing unit 280 includes a search unit 281, a dietary supplement determination unit 282, a physical change calculator 283, an inference unit 284, a first comparator 285, a generator 286, a second comparator 287, and an assessment unit 288.

The search unit 281 searches the nutrient information memory 12I for nutrient information that has a correlation with information relating to exercise to be performed by the user and also has a correlation with an objective relating to health. For example, the search unit 281 can search the nutrient information memory 12I (a nutrient information table 562 to be described later) and acquire nutrient information “vitamin B1”, “L-carnitine”, and the like to have a correlation with both “running”, that is the information relating to exercise to be performed by the user, and “dieting” that is the objective relating to health.

The dietary supplement determination unit 282 determines types of nutrients and compounding amounts of the nutrients based on the nutrient information obtained by the search unit 281. The dietary supplement determination unit 282 constitutes the calculator.

The physical change calculator 283 calculates, based on physical information of the user, lifestyle habit information, and biological information and external environment information acquired by the sensor 26, an amount of change in the physical information of the user before and after the user engages in exercise and takes in the dietary supplement.

For example, when the user engages in exercise and takes in a dietary supplement over a certain period of time, the physical change calculator 283 compares measured values of body-fat percentage or muscle mass before and after the period while taking external factors into consideration and calculates an effect of the exercise and the dietary supplement intake on the body-fat percentage and the muscle mass. Values obtained by body composition measurement can be used as the body-fat percentage, the muscle mass, and the like. An analysis can be performed to determine which exercise or dietary supplement most contributes to reducing the body-fat percentage or increasing the muscle mass at each body site, based on the amount of change in the physical information calculated by the physical change calculator 283.

The inference unit 284 infers, based on information on the time point at which the first acquisition unit 271 has acquired the information relating to exercise to be performed by the user, time during which the user has engaged in the exercise. Specifically, when a user uses more than one fitness device 300 at a facility such as a sports gym, the time elapsed between the start of use of one device and the start of use of the next device is inferred as the duration during which the user engaged in exercise with the first fitness device 300.

For example, the inference unit 284 can infer that the time elapsed between when the first acquisition unit 271 read the two-dimensional code 30 of the treadmill and a when the first acquisition unit 271 read the two-dimensional code 30 of a chest press used next by the user represents the duration the user spent running on the treadmill.

The first comparator 285 compares information relating to exercise and an amount of change in physical information among a plurality of users whose physical characteristics have a value within a certain range from a value of physical characteristics of the user, and who have received a supply of a dietary supplement. In addition to physical information such as the weight, body-fat percentage, and muscle mass of the user that may change due to exercise or dietary supplement intake, the physical characteristics of the user include characteristics such as height, sex, and age that generally remain unchanged regardless of exercise or nutritional intake. Dietary supplements taken in by the plurality of users may be the same nutrient.

The first comparator 285 refers to the physical information memory 12J and groups a plurality of users whose age, height, sex, weight, body-fat percentage, muscle mass, or the like is similar, and who have also received a predetermined dietary supplement, and compares a frequency or types of exercise performed among the users in a set time period. The first comparator 285 may further group a plurality of users who share the same objective relating to health and perform the comparison described above.

Based on the result of the comparison by the first comparator 285, the generator 286 generates exercise assistance information for the exercise to be performed by the user in accordance with the objective relating to health of the user. For example, suppose that, a user A has lost 1 kg in weight in a set time period such as three months, and another user B has lost 3 kg, and the user A was using the treadmill for 10 times while the user B was using the treadmill for 40 times. Based on this comparison, the generator 286 can generate exercise assistance information, such as “increase the frequency of using the treadmill” to assist the user A's dieting goal.

The second comparator 287 compares an amount of change in physical information of a user having received a supply of a dietary supplement by the dietary supplement supply device 400 (a supplier) with an amount of change in physical information of a user not having received a supply of the dietary supplement. For example, the second comparator 287 compares an amount of change in physical information such as weight, body-fat percentage, or muscle mass between the user A having taken a specific dietary supplement and the user B not having taken the dietary supplement in a set period such as three months. When making the comparison, the second comparator 287 may group and compare users having similar physical characteristics such as height, weight, and age.

The assessment unit 288 calculates and assesses the degree of contribution of a supply of the dietary supplement to the amount of change in the physical information of the user who has received the supply of the dietary supplement, based on the comparison results from the second comparator 287. For example, the assessment unit 288 calculates how much the supply of a specific dietary supplement has contributed to the amount of change in physical information such as weight, body-fat percentage, or muscle mass of the user as the degree of contribution. The assessment unit 288 can calculate the degree of contribution by a regression analysis, an analysis of variance, machine learning, or the like. In addition, the assessment unit 288 assesses the calculated degree of contribution and analyzes which dietary supplement supply may have a particular effect on the amount of change in physical information of the user.

### [Functional Blocks of Dietary Supplement Supply Device]

FIG. 5 shows a functional block diagram of the dietary supplement supply device 400 with the hardware configuration described above. The dietary supplement supply device 400 includes an interface 420, a supply unit 440 which carries out an operation for supplying a dietary supplement, and a dietary supplement compounding assistance unit 460.

The interface 420 includes, in addition to an input/output 421 and a transmission/reception unit 422, a command unit 423 which provides a supply command to the supply unit 440 based on a supply instruction from the user device 200, a usage history transmitting unit 424 which transmits a usage history to the management server 100 or the user device 200 through the network 2, a user identification unit 425 which is necessary when using the dietary supplement supply device 400 offline, and a user authenticator 426 which authenticates user information.

The supply unit 440 includes a slot 441 to which a cartridge storing a dietary supplement is mounted and a dietary supplement output 445 which extracts and compounds a dietary supplement from a cartridge and externally supplies the dietary supplement.

The slot 441 includes a cartridge information reader (a reader) 442 which reads cartridge-related information of the mounted cartridge, and a remaining dietary supplement amount identification unit 443 which identifies the remaining amount of the dietary supplement stored in the cartridge.

The dietary supplement output 445 includes a driver 446 which drives a motor and rotates a cartridge in order to extract a predetermined amount of a dietary supplement in accordance with a command from the command unit 423 and a dispense processing unit 447 which operates and actually compound the extracted dietary supplement, and dispense the dietary supplement externally. The dietary supplement compounding assistance unit 460 (dashed line part) is similar to the dietary supplement compounding assistance unit 260 shown in FIG. 4.

It should be noted that, in the dietary supplement compounding assistance system 1 according to the present embodiment, a computer to execute logic relating to processing of compounding and supplying dietary supplements can be optionally selected from information processing apparatuses and the like including the management server 100, the user device 200, and the dietary supplement supply device 400 described above. In a similar manner, a computer to execute logic to search for nutrient information including a correlation with information relating to exercise to be performed by the user and a correlation with an objective relating to health and to determine types and compositions of nutrients based on the searched-for nutrient information can also be optionally selected from information processing apparatuses and the like including the management server 100, the user device 200, and the dietary supplement supply device 400 described above.

In other words, a set of programs and modules that causes the dietary supplement compounding assistance units 160, 260, and 460 to function can be stored in any of the elements described above and can be executed by the processor of any of the elements described above. In addition, all of the set of programs and modules may be arranged in any of the elements described above in a centralized manner or a part of the set of programs and modules may be arranged in a distributed manner.

As an example, a main part of the set of programs and modules can be arranged in the user device 200 and can determine a dietary supplement to be supplied by communicating with the management server 100 and the dietary supplement supply device 400. As another example, all of the set of programs and modules may be arranged in the dietary supplement supply device 400 in a centralized manner, in which case the dietary supplement supply device 400 is to function offline as a stand-alone apparatus.

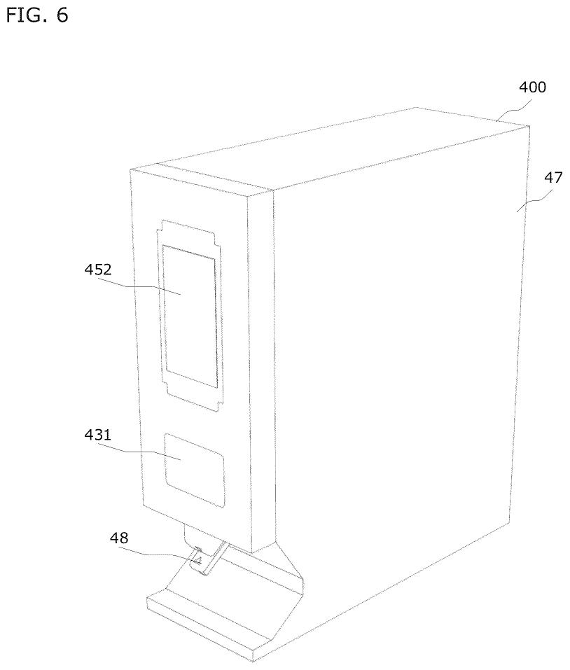

Next, the structure of the dietary supplement supply device 400 will be described in greater detail with reference to FIGS. 6 and 7. FIGS. 6 and 7 show a structure of an enclosure that realizes the dietary supplement supply device 400. FIG. 6 shows an exterior of an exemplary enclosure of the dietary supplement supply device 400. In the exterior of the enclosure example, the compounding unit 47 occupies a significant portion of the enclosure. A display 452 is provided on a front surface and the user authenticator 426 is located below the display 452, and the dispenser 48 is placed beneath the user authenticator 426.

The processor 40, the memory 41, the storage 42, the communication interface 43, and the like are incorporated inside the enclosure and the sensor 46 is mounted on a surface part (none of which are illustrated) of the enclosure. The sensor 46 is provided as, for example, a sensor electrode and can acquire biological information, such as heart rate, by coming into contact with the user, as well as environmental information, such as air temperature and humidity.

A user authenticator 426 has, for example, a built-in camera or the like and reads user information including a user ID indicated by a two-dimensional code such as a barcode displayed on the display 252 of the user device 200. The dispenser 48 is provided with a discharge chute extending downward, and by placing a container such as a cup below the discharge chute, the user can receive an output of a compounded dietary supplement in a tablet form from a dispensing port provided in an upper part.

FIG. 7 shows a schematic structure of the inside of the compounding unit 47 shown in FIG. 6. The compounding unit 47 with a multistage structure includes the slot 441 to which detachable cartridges 50 are mounted and the driver (motor) 446 that provides drive in accordance with a dietary supplement supply instruction. An IC chip 51 is provided on any surface such as a cartridge side surface or a bottom surface of each cartridge 50 and a discharge hole (not illustrated) for discharging a dietary supplement is provided at the rear of each cartridge 50. In addition, by providing a well-known tablet feeder adjacent to the slot 441 of the compounding unit 47 to which cartridges 50 are to be mounted, a dietary supplement in a tablet form can be dispensed. Furthermore, when dispensing a dietary supplement in a powder form, the dietary supplement can be dispensed by rotating an auger part of a spiral structure (not illustrated) adjacent to the slot 441 to which the cartridges 50 are to be mounted. Note that a method of dispensing a dietary supplement is not limited to using a motor and, for example, a configuration may be adopted in which an on-off valve is provided in a bottom part of each cartridge 50 and a dietary supplement is dispensed by driving the on-off valve with a solenoid.

The cartridge information reader 442 of the slot can function as a reader for the IC chip 51 and read various pieces of information from the cartridges 50. Specifically, each cartridge has one or more IDs (for example, a cartridge ID and a stored dietary supplement ID), an initially stored dietary supplement amount, a dietary supplement discharge amount, and the like stored in a memory and the cartridge information reader 442 can read these pieces of information. In addition, the remaining dietary supplement amount identification unit 443 of the slot can identify the remaining amount inside the cartridge in accordance with the location of slot.

For example, but not limited thereto, the remaining amount inside the cartridge 50 can be associated with the number of rotations of the motor of the driver 446 and an accumulated number of rotations of the motor is to be stored in a memory of the IC chip 51. In addition, after inserting a cartridge, the accumulated number of rotations of the motor or, in other words, how many rotations the motor has made is read and by identifying the number of possible remaining rotations by comparing the accumulated number of rotations with the number of maximum remaining rotations that corresponds to the initial amount of a dietary supplement, the remaining dietary supplement amount at that time point is identified. In this manner, dietary supplement composition reference information is further adjusted by the dietary supplement determination unit 282 based on information including the remaining dietary supplement amount identified using the IC chip 51.

When the remaining amount of the dietary supplement using the IC chip 51 as described above is not detected, a capacity of the cartridge 50 is stored in advance by the supply unit 440, and the fact that the cartridge 50 has been replaced is input to the dietary supplement supply device 400 via an input 451 or when there is no need to detect a remaining amount of a dietary supplement, the IC chip 51 and a reader thereof described herein need not necessarily be provided. Note that the enclosure described above and shown in FIGS. 6 and 7 is merely illustrative and non-limiting.

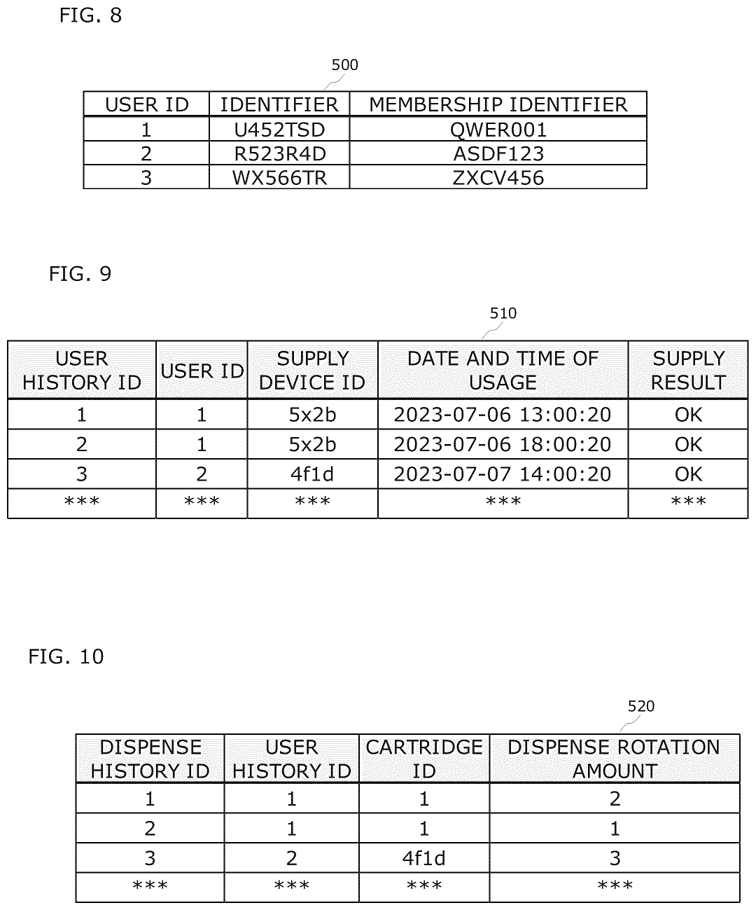

Next, FIGS. 8 to 18 show examples of table structures that constitute the various memories 12A to 12K included in the storage 12 of the management server 100. FIG. 8 shows a user information table 500 which is provided on the storage 12 and which realizes the user information memory 12A. In the user information table 500, an identifier of a user to receive a supply of a dietary supplement and a membership identifier of the user at a fitness facility such as a sports gym are associated with a user ID. Basic information of the user in the user information table 500 shown in FIG. 8 is managed by the user information manager 141. The basic information of the user is initially registered when the user subscribes to the service.

FIG. 9 shows a user history table 510 relating to a history of supply of dietary supplements of each user, and which corresponds to the user history memory 12B. Each time the user receives a supply of a dietary supplement, the user history manager 142 adds a record for each user history ID to the user history table 510 and stores and updates history information. A user ID, an ID of the dietary supplement supply device used, a date and time of usage, a supply result, and the like are associated with the user history ID.

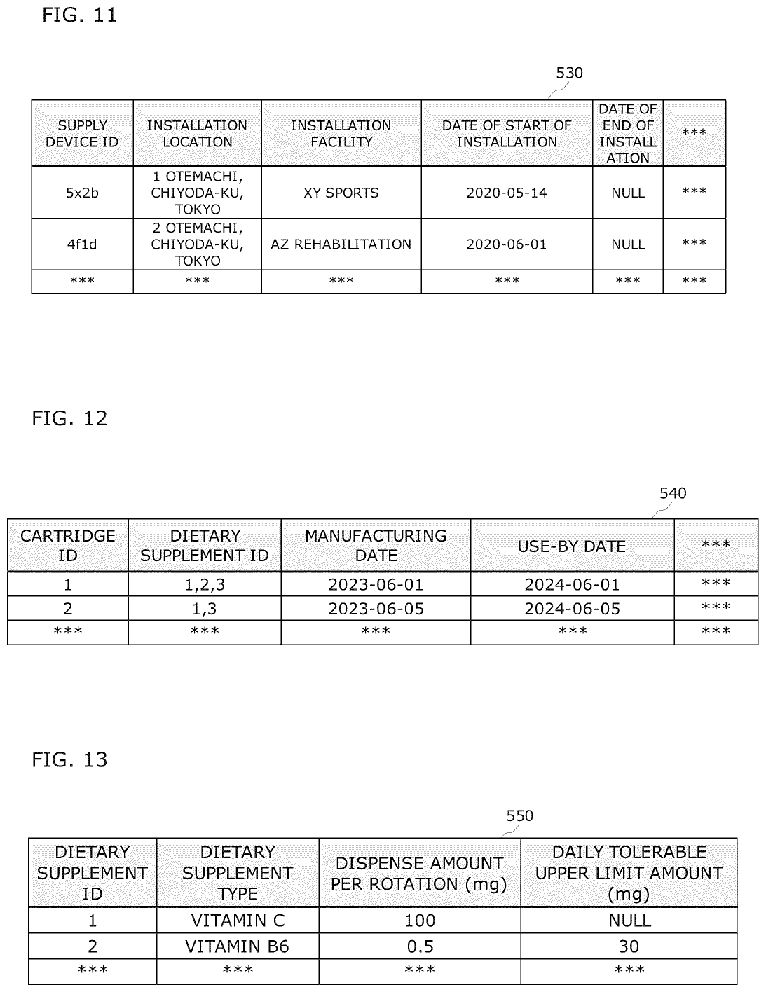

FIG. 10 shows a dispense history table 520 relating to a history of dietary supplement dispensation by the dietary supplement supply device 400, FIG. 11 shows a dietary supplement supply device management table 530, and the two tables correspond to the dietary supplement supply device memory 12C. In the dispense history table 520, a cartridge ID relating to a cartridge having dispensed a dietary supplement and a dispensation rotation amount which have been associated with a user history ID are stored for each dispense history ID. The dispense history table 520 can be updated by the user history manager 142.

In the dietary supplement supply device management table 530, the dietary supplement supply device manager 143 manages installation information of the dietary supplement supply device 400 to be installed in sports gyms, rehabilitation facilities, or the like. In other words, an installation location, an installation facility, a date of start of installation and a date of end of installation (null when continuously being used), and the like of the dietary supplement supply device are associated with respect to a unique dietary supplement supply device ID assigned to a dietary supplement supply device.

FIG. 12 shows a cartridge management table 540 (cartridge memory 12D) which stores cartridge information of the dietary supplement supply device 400, and FIG. 13 shows a dietary supplement management table 550 (tolerable upper limit amount memory 12F) which stores a daily tolerable upper limit amount of dietary supplements.

In the cartridge management table 540, information on cartridges that can be mounted on the dietary supplement supply device 400 is managed by the cartridge manager 144. With respect to a unique cartridge ID assigned to a cartridge, a stored dietary supplement ID of a dietary supplement stored inside the cartridge, a manufacturing date and time of the cartridge, and a use-by date and time of the cartridge are associated. An initial amount of the stored dietary supplement may be managed in association (not illustrated). Note that a cartridge ID and a stored dietary supplement ID need not be separate IDs. Which dietary supplement (cartridge) is attached to which slot (for example, the slot at the end) of the slot 441 in the installed dietary supplement supply device 400 can be optionally selected by a maintenance administrator of the dietary supplement supply device 400.

In the dietary supplement management table 550, a dietary supplement type associated with the dietary supplement ID which uniquely identifies a dietary supplement is managed by the dietary supplement compounding assistance manager 145. For example, “dietary supplement type: vitamin C” is associated with “dietary supplement ID: 1”. In addition, an extraction amount per rotation of a cartridge of the dietary supplement type is also identified. This is because each dietary supplement has a different powder density and the number of rotations and the extraction amount (mg) must be associated with each dietary supplement type. As described with reference to FIGS. 6 and 7, when a cartridge ID is read through the IC chip included in a cartridge, the dietary supplement supply device 400, the user device 200, or the management server 100 identifies the cartridge mounted to the slot 441 by referring to the cartridge management table 540 and the dietary supplement management table 550.

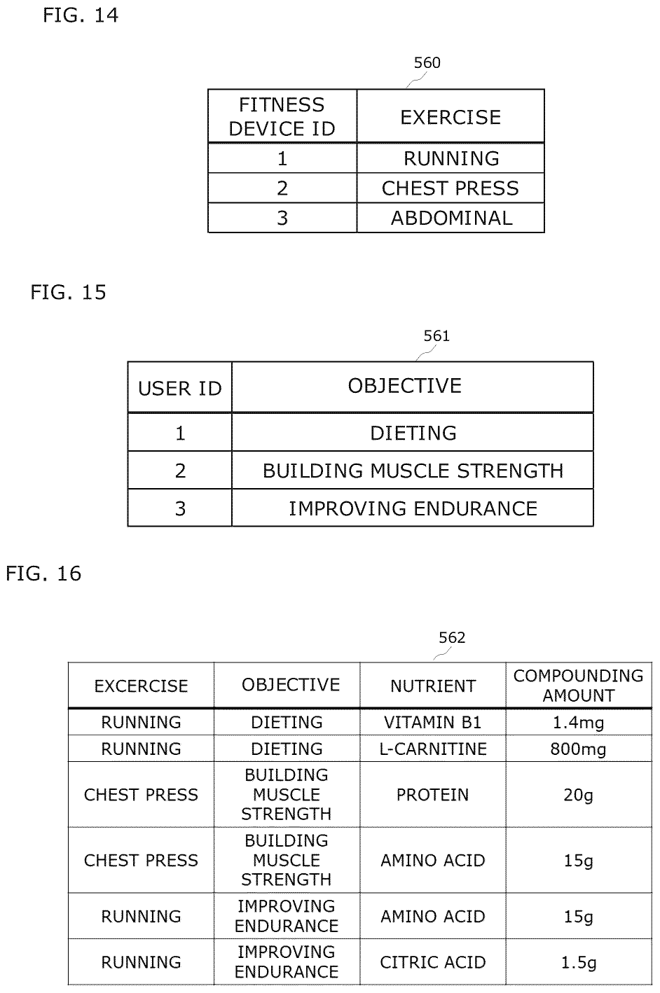

FIG. 14 shows the exercise information table 560 (exercise information memory 12G) in which an ID of a fitness device and a type of exercise are associated with each other. In the exercise information table 560, the exercise type associated with the fitness device ID is managed by the exercise information manager 147.

FIG. 15 shows an objective information table 561 (objective information memory 12H) in which the user's objective relating to health, input by the user, is associated with each user ID. In the objective information table 561, an objective relating to health which is set for each user is managed by the objective information manager 148.

FIG. 16 shows the nutrient information table 562 (nutrient information memory 12I) in which nutrient information that has a correlation with a type of exercise and a correlation with an objective relating to health of the user is registered. In the nutrient information table 562, nutrients having a correlation with exercise and a correlation with an objective relating to health of the user are managed by the nutrient information manager 149. More specifically, the nutrient information table 562 can be generated for each user ID and, as shown in FIG. 16, an exercise, an objective relating to health, nutrients, and compounding amounts of the nutrients can be registered in association with each user ID. In addition to nutrient information relating to correlation prepared in advance, as will be described later, correlation information in the nutrient information table 562 is updated based on a review (assessment) relating to a supply of a dietary supplement that is input by a user after the dietary supplement is supplied to the user.

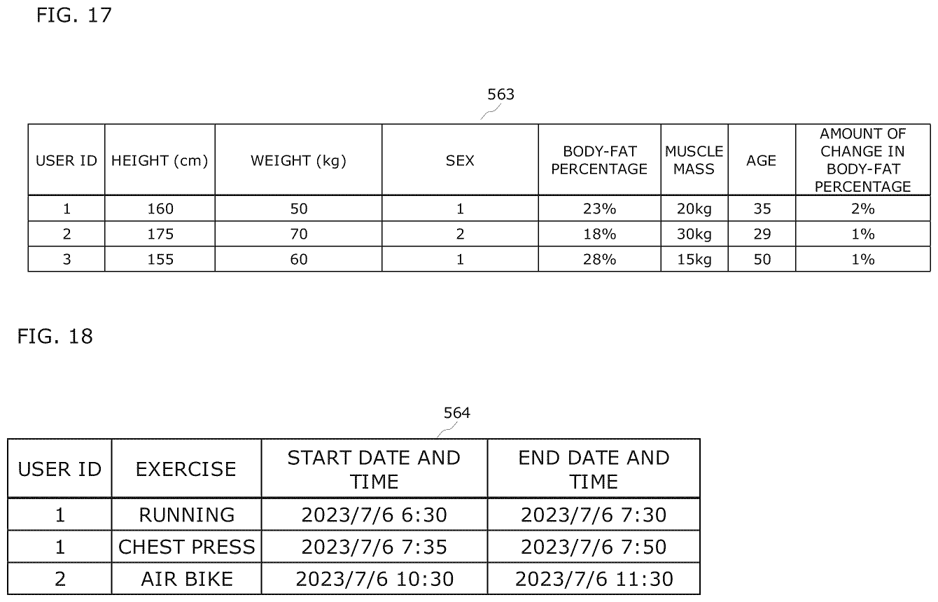

FIG. 17 shows a physical information table 563 (physical information memory 12J) in which physical characteristics and physical information of the user such as height, weight, sex, body-fat percentage, muscle mass, age, an amount of change in body-fat percentage, and the like and an amount of change in the physical information are stored in association with each user ID. The physical information table 563 is managed by the physical information manager 150. Note that in addition to the above, information relating to lifestyle habits, such as whether or not the user smokes, an amount of sleep, an amount of activity, and the like can be associated with the user ID in the physical information table 563.

FIG. 18 shows an exercise history table 564 (exercise history memory 12K) which stores a start date and time and an end date and time of each exercise for each user ID. In the exercise history table 564, information on the time point at which the user had engaged in each exercise is managed by the exercise history manager 151.

The group of tables is mainly managed in the memory 11 or the storage 12 of the management server 100. In addition, the group of tables can be also stored in the user device 200 and the dietary supplement supply device 400 in synchronization with the management server 100. In other words, the group of tables can be made storable in any of the elements described above and can be made locally and/or globally referable by each processor. Furthermore, a set of the group of tables may be partially and optionally arranged in distributed manner. Note that the group of tables described above is merely illustrative and, as will be appreciated by those skilled in the art, the group of tables is not intended to be limited to the configurations, types, items, data contents, data types, and the like described herein.

### [Operation of Dietary Supplement Compounding Assistance System]

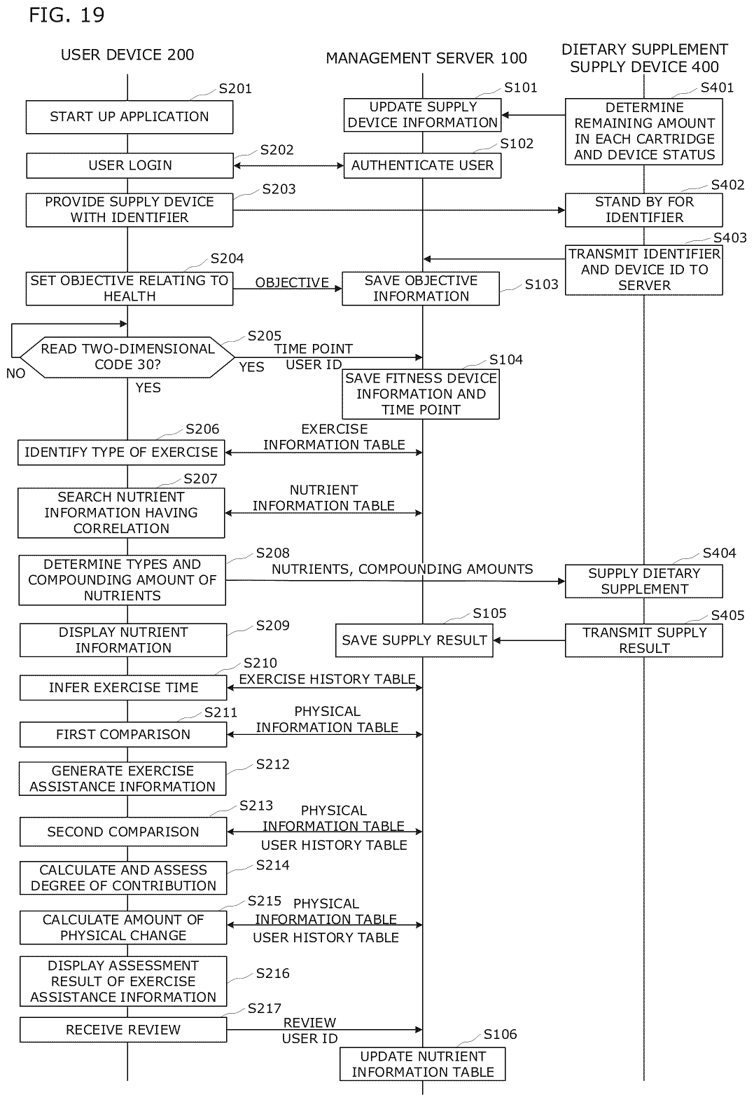

Next, the operation of the dietary supplement compounding assistance system 1 configured as described above will be explained with reference to a sequence diagram shown in FIG. 19.

First, as shown in FIG. 19, the remaining dietary supplement amount identification unit 443 of the dietary supplement supply device 400 determines a remaining amount in each cartridge and a status of the dietary supplement supply device 400 (Step S401). The determination result is transmitted from the transmission/reception unit 422 of the dietary supplement supply device 400 to the management server 100 via the network 2. Next, the dietary supplement supply device manager 143 of the management server 100 updates dietary supplement supply device information in the dietary supplement supply device management table 530 based on the remaining amount of each cartridge and the status of the dietary supplement supply device 400 received by the transmission/reception unit 123 in step S401 (Step S101).

On the other hand, an application starts up on the user device 200 (Step S201), the user authenticator 241 refers to the user information table 500, and the user authenticator 241 carries out login processing with the user information manager 141 of the management server 100 (Step S202 and Step S102). More specifically, login processing can be performed by allowing the user authenticator 426 of the dietary supplement supply device 400 to read a barcode containing unique information for the user which is displayed on the display 252 on the user device 200.

As alternatives to a barcode, any login processing method can be adopted including wireless authentication using BLE and authentication using one of the display screen (input/output 421) of the dietary supplement supply device 400 and the display 252 of the user device 200 to display a one-time passcode and having the counterpart device input the one-time passcode. The user login in Step S202 is login processing to a service provided by the dietary supplement compounding assistance system 1.

The input/output 222 of the user device 200 provides the dietary supplement supply device 400 with an identifier of the user (Step S203). For example, the user device 200 can provide the dietary supplement supply device 400 installed in a fitness facility with data containing an identifier by near field communication such as BLE or the like. The user identification unit 425 and the user authenticator 426 of the dietary supplement supply device 400 having received the identifier of the user identify and authenticate the user (Step S402). Subsequently, the transmission/reception unit 422 of the dietary supplement supply device 400 transmits the identifier of the user and the device ID of the dietary supplement supply device 400 to the management server 100 (Step S403).

Next, the second acquisition unit 272 of the user device 200 receives an input operation of an objective relating to health of the user from the input/output 222, and sets the objective relating to health (Step S204). The objective information table 561 of the management server 100 saves the health related objective of the user acquired by the second acquisition unit 272 (Step S103).

Next, the user uses the fitness device 300 at a sports gym or the like. When the user uses the fitness device 300, the first acquisition unit 271 of the user device 200 activates the camera 27 to read the two-dimensional code 30 provided on the fitness device 300 (Step S205: YES), the ID of the fitness device 300 and a start time are saved in the exercise history table 564 of the management server 100 (Step S104).

Each time the user switches the fitness device 300 and engages in different exercise, the first acquisition unit 271 of the user device 200 reads the two-dimensional code 30 of the fitness device 300 used by the user and the time at which the code is read is saved in the exercise history table 564 as an exercise start time or an exercise end time.

Next, the first acquisition unit 271 of the user device 200 refers to the exercise information table 560 based on the two-dimensional code 30 read in Step S205 and identifies a type of exercise corresponding to the two-dimensional code 30 (Step S206). Next, the search unit 281 of the user device 200 searches the nutrient information table 562 for nutrient information that has a correlation with the objective relating to health of the user set in Step S204 and has a correlation with the type of exercise identified in Step S206 (Step S207).

Next, the dietary supplement determination unit 282 determines types of nutrients and compounding amounts of the nutrients based on the nutrient information searched for and obtained in Step S207 (Step S208). The determined types of nutrients and compounding amounts of the nutrients are sent to the dietary supplement supply device 400. Subsequently, the supply unit 440 of the dietary supplement supply device 400 supplies a dietary supplement containing the determined types of nutrients and compounding amounts (Step S404). Furthermore, the dietary supplement supply device 400 transmits a supply result of the dietary supplement to the management server 100 from the interface 420 (Step S405).

The management server 100 having received the supply result saves the supply result in the user history table 510 (Step S105). On the other hand, the display 252 of the user device 200 is triggered to display the nutrient information relating to the supply of the dietary supplement (Step S209).

Subsequently, the inference unit 284 of the user device 200 infers a time during which the user had engaged in exercise based on information regarding the time point at which the two-dimensional code 30 of the fitness device 300 was read in Step S205 (Step S210). Specifically, the inference unit 284 refers to the exercise history table 564 and infers a time elapsed between when the two-dimensional code 30 of the first fitness device 300, which the user used to engage in exercise, was read and when the two-dimensional code 30 of the second fitness device 300 was read, as the duration the user had engaged in exercise using the first fitness device 300.

Next, the first comparator 285 of the user device 200 compares information relating to exercise and an amount of change in physical information among a plurality of users, who have received a supply of a dietary supplement, and whose physical characteristics have a value within a certain range from a value of physical characteristics of the user (Step S211).

Specifically, the first comparator 285 refers to the physical information table 563, the objective information table 561, the user history table 510, and the exercise history table 564 and groups a plurality of users having physical characteristics such as height, weight, age, sex, and the like that are similar to those of the user. Additionally, the first comparator 285 can obtain a comparison result indicating that, among users receiving the same or similar dietary supplement, user A has achieved a significantly reduced body-fat percentage because user A was running more frequently compared to user B who did not participate in running at all.

Next, based on the result of the comparison in Step S211, the generator 286 generates exercise assistance information in accordance with the objective relating to health of the user (Step S212). For example, according to the result of the comparison in Step S211, when the user B's health related objective is dieting, the generator 286 can generate information indicating the use of a treadmill and a frequency of use thereof or the use of a stair stepper which provides a similar exercise effect and a frequency of use thereof as the exercise assistance information.

Next, the second comparator 287 of the user device 200 compares the amount of change in physical information of a user who has received the dietary supplement by the dietary supplement supply device 400 with the amount of change in physical information of a user who did not receive the dietary supplement (Step S213). Specifically, the second comparator 287 refers to the physical information table 563 and the user history table 510 and compares the amount of change in the body-fat percentage or the like of the user A having been supplied a specific dietary supplement with the amount of change in the body-fat percentage or the like of the user B not having been supplied the dietary supplement.

Next, based on a result of the comparison in Step S213, the assessment unit 288 calculates and assesses the degree of contribution of the supply of the specific dietary supplement to the amount of change in physical information of the user having received a supply of the dietary supplement (Step S214).

Next, the physical change calculator 283 calculates, based on physical information of the user, lifestyle habit information, and biological information and external environment information acquired by the sensor 26, an amount of change in the physical information of the user before and after the user engages in exercise and takes in the dietary supplement (Step S215). The physical change calculator 283 refers to the physical information table 563, the user history table 510, and the exercise history table 564 and, after eliminating external factors, calculates the amount of change in the body-fat percentage or the like before and after being supplied the dietary supplement and engaging in exercise. Effects of the supply of the dietary supplement and the exercise on the user's physical information can be analyzed based on such an amount of change in physical information.

Next, the display 252 of the user device 200 displays the exercise assistance information generated in Step S212, the degree of contribution by the supply of the dietary supplement and the assessment of the degree of contribution obtained in Step S214, and the amount of change in the physical information obtained in Step S215 (Step S216).

Next, the input/output 222 of the user device 200 receives a review from the user who has received the dietary supplement supply service (Step S217). The review received in Step S217 is an assessment regarding a subjective view of their own physical shape, mood, satisfaction, and the like of the user who has received the supply of the dietary supplement by the dietary supplement compounding assistance system 1, which considers both aspects of exercise and nutrition.

The review by the user received in Step S217 is fed back to the nutrient information table 562, and the nutrient information that has the correlation with exercise and the objective relating to health, is updated (Step S106). As described earlier, the nutrient information table 562 can be provided for each user. In this case, based on a review by each user received in Step S217, the nutrient information table 562 can be updated for each user in Step S106. Thus, when the search unit 281 searches for nutrient information having a correlation with exercise and an objective relating to health in Step S207, the search is performed based on correlation reflecting the user review received in Step S217.

As described above, according to the dietary supplement compounding assistance system 1 of the present embodiment, nutrient information having a correlation with exercise to be performed by a user and an objective relating to health is searched for from the nutrient information table 562, and types of nutrients and compounding amounts of the nutrients are determined based on the searched-for and obtained nutrient information. Thus, more effective assistance can be achieved for compounding dietary supplements based on the correlation between exercise and nutrition in order to achieve and enhance health of each user.

In addition, the dietary supplement compounding assistance system 1 according to the present embodiment compares information relating to exercise and dietary supplement intake information among users, and therefore capable of presenting exercise assistance information tailored to each user's health related objective.

In the embodiment, an example was provided to illustrate that the first acquisition unit 271 acquires information relating to exercise to be performed by a user by reading the two-dimensional code 30 of the fitness device 300 having been captured by the camera 27. However, a method used by the first acquisition unit 271 to acquire information relating to exercise is not limited to reading the two-dimensional code 30. For example, the second acquisition unit 272 may acquire information relating to exercise by performing image recognition on images captured by a camera installed at a sports gym or a rehabilitation facility where the fitness device 300 is installed and performing object recognition of a user and the fitness device 300 used by the user. For example, information relating to exercise can be acquired by performing object detection of the user and the fitness device 300 by deep learning such as R-CNN, SSD, YOLO, or Fast R-CNN and by inferring a class and a position of the detected objects.

In the embodiment, an example was provided to illustrate that the two-dimensional code 30 is attached to the fitness device 300. However, alternative configurations can be used as long as they allow for the acquisition of information relating to the exercise performed by the user. For example, the two-dimensional code 30 installed at a studio, a fitness facility such as a swimming pool, or a rehabilitation facility where the type of exercise can be identified, can be utilized. Additionally, a method of acquiring information relating to exercise is also not limited to reading the two-dimensional code 30 and the information can be acquired by near field communication such as BLE, NFC, or RFID.

According to the dietary supplement compounding assistance system 1 of the present embodiment, a user does not need to perform an input operation each time the fitness device 300 is used. This allows for more efficient operation of the dietary supplement compounding assistance system as the system's usability is improved.

Further, in the embodiment, an example was provided to illustrate that the presenter is constituted of the display 252 and nutrient information, time information, assessment results, and the like are output as visual information. However, the presenter is not limited to be configured as the display 252, and information can be presented by sound, a combination of visual information and vibration, or the like.

While there is described herein the embodiment of the dietary supplement compounding assistance system and the dietary supplement compounding assistance method according to the present disclosure, the present disclosure is not limited to the described embodiments and various modifications that could be conceived by those skilled in the art may be made within the scope of the invention described in the claims.

### REFERENCE SIGNS LIST

1 dietary supplement compounding assistance system, 2 network, 10, 20, 40 processor, 11, 21, 41 memory, 12, 22, 42 storage, 13, 23, 43 communication interface, 14, 24, 44 input/output (I/O), 25, 45 touch panel, 26, 46 sensor, 47 compounding unit, 48 dispenser, 251, 451 input, 252, 452 display, 100 management server, 200 user device, 300 fitness device, 400 dietary supplement supply device.

What is claimed is:

1. A dietary supplement compounding assistance system, comprising:
a first acquisition unit which acquires information relating to exercise to be performed by a user;
a second acquisition unit which acquires an objective relating to health of the user;
a first memory which stores nutrient information having a correlation with the information relating to exercise, and having a correlation with the objective relating to health;
a search unit which searches the first memory for nutrient information having a correlation with the information relating to exercise, and having a correlation with the objective relating to health;
a calculator which determines a type of nutrient and a compounding amount of the nutrient based on the searched-for and obtained nutrient information;
a presenter which presents the searched-for and obtained nutrient information;
a supplier which supplies a dietary supplement containing the compounding amount of the nutrient determined by the calculator; and
a physical change calculator which calculates, based on physical information of the user, an amount of change in the physical information of the user before and after the user engages in the exercise and takes in the dietary supplement, wherein
the presenter further presents information relating to a supply of the dietary supplement and an amount of change in the physical information of the user.

2. The dietary supplement compounding assistance system according to claim 1, wherein
the physical change calculator further calculates, based on lifestyle habit information of the user, biological information or external environment information acquired by a sensor, and a combination of these pieces of information, an amount of change in the physical information of the user before and after the user engages in the exercise and takes in the dietary supplement.

3. The dietary supplement compounding assistance system according to claim 2, further comprising:
a second memory which stores the information relating to the exercise of the user, the objective relating to health, information relating to a supply of the dietary supplement, the amount of change in the physical information, and physical characteristics of the user in association with identification information of the user;
a first comparator which compares the information relating to exercise and the amount of change in the physical information among a plurality of users whose physical characteristics have a value within a certain range from a value of the physical characteristics of the user, and who have received a supply of the dietary supplement; and
a generator which generates exercise assistance information for the exercise to be performed by the user in accordance with the objective relating to health of the user based on a result of the comparison by the first comparator, wherein
the presenter presents the exercise assistance information to the user.

4. The dietary supplement compounding assistance system according to claim 3, further comprising:
a second comparator which compares between an amount of change in the physical information of the user having received a supply of the dietary supplement by the supplier and an amount of change in the physical information of a user not having received a supply of the dietary supplement; and
an assessment unit which calculates, based on a result of the comparison by the second comparator, a degree of contribution of a supply of the dietary supplement relative to the amount of change in the physical information of the user having received the supply of the dietary supplement, and which assesses the degree of contribution, wherein
the presenter presents a result of the assessment by the assessment unit.

5. The dietary supplement compounding assistance system according to claim 4, further comprising:
an inference unit which infers time during which the user had engaged in the exercise based on information on a time point at which the first acquisition unit had acquired the information relating to exercise, wherein
the presenter presents the inferred time during which the user had engaged in the exercise.

6. The dietary supplement compounding assistance system according to claim 5, further comprising:
a reception which receives a review relating to a supply of the dietary supplement from the user having received a supply of the dietary supplement.

7. The dietary supplement compounding assistance system according to claim 6,
wherein the search unit searches the first memory for the nutrient information in consideration of the review received by the reception.

8. A dietary supplement compounding assistance method executed by a dietary supplement compounding assistance system, the dietary supplement compounding assistance method comprising:
a first step of acquiring information relating to exercise to be performed by a user;
a second step of acquiring an objective relating to health of the user;
a third step of storing, in a first memory, nutrient information having a correlation with the information relating to exercise, and having a correlation with the objective relating to health;
a fourth step of searching the first memory for nutrient information having a correlation with the information relating to exercise, and having a correlation with the objective relating to health;
a fifth step of determining a type of nutrient and a compounding amount of the nutrient based on the searched-for and obtained nutrient information;
a sixth step of presenting the searched-for and obtained nutrient information;
a seventh step of supplying a dietary supplement containing the compounding amount of the nutrient determined in the fifth step; and
an eighth step of calculating, based on physical information of the user, an amount of change in the physical information of the user before and after the user engages in the exercise and takes in the dietary supplement, wherein
in the sixth step, information relating to the supply of the dietary supplement and the amount of change in the physical information of the user are further presented.

9. The dietary supplement compounding assistance method according to claim 8, wherein
in the eighth step, based on lifestyle habit information of the user, biological information or external environment information acquired by a sensor, and a combination of these pieces of information, an amount of change in the physical information of the user before and after the user engages in the exercise and takes in the dietary supplement is further calculated.

10. The dietary supplement compounding assistance method according to claim 9, further comprising:
a ninth step of storing, in a second memory, the information relating to the exercise of the user, the objective relating to health, information relating to a supply of the dietary supplement, the amount of change in the physical information, and physical characteristics of the user in association with identification information of the user;
a tenth step of comparing the information relating to exercise and the amount of change in the physical information among a plurality of users whose physical characteristics have a value within a certain range from a value of the physical characteristics of the user, and who have received a supply of the dietary supplement; and
an eleventh step of generating exercise assistance information for the exercise to be performed by the user in accordance with the objective relating to health of the user based on a result of the comparison in the tenth step, wherein
in the sixth step, the exercise assistance information is presented to the user.

11. The dietary supplement compounding assistance method according to claim 10, further comprising:
a twelfth step of comparing between an amount of change in the physical information of the user having received a supply of the dietary supplement in the seventh step and an amount of change in the physical information of a user not having received a supply of the dietary supplement; and
a thirteenth step of calculating a degree of contribution of a supply of the dietary supplement relative to the amount of change in the physical information of the user having received the supply of the dietary supplement based on a result of the comparison in the twelfth step, and assessing the degree of contribution, wherein
in the sixth step, a result of the assessment in the thirteenth step is presented.

12. The dietary supplement compounding assistance method according to claim 11, further comprising:
a fourteenth step of inferring time during which the user had engaged in the exercise based on information on a time point at which the information relating to exercise acquired in the first step, wherein
in the sixth step, the inferred time during which the user had engaged in the exercise is presented.

13. The dietary supplement compounding assistance method according to claim 12, further comprising:
a fifteenth step of receiving a review relating to a supply of the dietary supplement from the user having received the supply of the dietary supplement.

14. The dietary supplement compounding assistance method according to claim 13, wherein
in the fourth step, the nutrient information is searched for from the first memory in consideration of the review received in the fifteenth step.
